(19) United States
(12) Patent Application Publication
(10) Pub. No.: US 2015/0299160 A1
(43) Pub. Date: Oct. 22, 2015

(54) PROCESS FOR THE PREPARATION OF RIVAROXABAN AND INTERMEDIATES THEREOF

(71) Applicant: RANBAXY LABORATORIES LIMITED, New Delhi, Delhi (IN)

(72) Inventors: Pankaj Kumar SINGH, Kanpur (IN); Mohammed Salman HASHMI, Aligarh (IN); Yoginder Pal SACHDEVA, Firozepur (IN); Chandra Has KHANDURI, Gurgaon (IN)

(21) Appl. No.: 14/394,547

(22) PCT Filed: Apr. 16, 2013
(86) PCT No.: PCT/IB2013/053025

§ 371 Date: Oct. 15, 2014

(30) Foreign Application Priority Data
Apr. 16, 2012 (IN) 1174/DEL/2012

(51) Int. Cl. C07D 333/38; C07D 409/12; C07D 413/12; C07D 413/14

(52) U.S. Cl. CPC C07D 333/38; C07D 413/14; C07D 409/12; C07D 413/12

(57) ABSTRACT

The present invention provides processes for the preparation of rivaroxaban and its intermediates.

### FIELD OF THE INVENTION

The present invention provides processes for the preparation of rivaroxaban and its intermediates.

### BACKGROUND OF THE INVENTION

Rivaroxaban chemically is 5-chloro-N-({(5S)-2-oxo-3-[4-(3-oxomorpholin-4-yl)phenyl]-1,3-oxazolidin-5-yl}methyl)thiophene-2-carboxamide of Formula I.

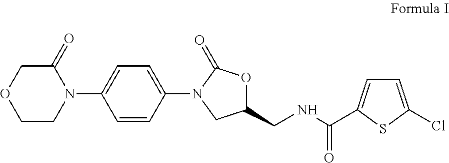

Rivaroxaban is used as an anti-thrombotic agent.

U.S. Pat. No. 7,157,456 provides rivaroxaban and processes for its preparation.

U.S. Pat. No. 8,106,192 provides a process for the preparation of N—((S)-3-bromo-2-hydroxypropyl)-5-chlorothiophene-2-carboxamide, wherein (2S)-3-aminopropane-1,2-diol hydrochloride is reacted with 5-chlorothiophene-2-carbonyl chloride to provide N—((S)-2,3-dihydroxypropyl)-5-chlorothiophene-2-carboxamide. The resulting compound is treated with hydrobromic acid in the presence of acetic anhydride at 60° C. to 65° C., and the reaction mixture is stirred overnight to give N—((S)-3-bromo-2-hydroxypropyl)-5-chlorothiophene-2-carboxamide, which is further converted into rivaroxaban.

U.S. Publication No. 2010/0273789 provides a process for the preparation of 5-chloro-N-[(2S)-oxiran-2-ylmethyl]thiophene-2-carboxamide, wherein ((S)-3-bromo-2-hydroxypropyl)-5-chlorothiophene-2-carboxamide (50 g, 0.167 moles) is stirred with potassium carbonate (155 g, 1.12 moles) in the presence of anhydrous tetrahydrofuran (500 mL) for three days at room temperature to give 5-chloro-N-[(2S)-oxiran-2-ylmethyl]thiophene-2-carboxamide.

U.S. Publication No. 2007/0066615 provides a process for the preparation of 5-chloro-N-((2R)-2-hydroxy-3-{[4-(3-oxo-4-morpholinyl)-phenyl]amino}propyl)-2-thiophenecarboxamide, wherein a solution of 4-(4-aminophenyl)morpholin-3-one (2.6 mmol) and 5-chloro-N-[(2S)-oxiranylmethyl]-2-thiophenecarboxamide (3.1 mmol) in tetrahydrofuran is stirred overnight at 60° C. in the presence of ytterbium(III) trifluoromethanesulfonate to give a precipitate, which is filtered off to provide the product in 54% yield. The remaining filtrate is concentrated and the residue obtained is purified by preparative HPLC to provide further 38% of the product.

U.S. Publication No. 2010/0120718 provides a general method for preparing substituted N-(3-amino-2-hydroxypropyl)-5-chloro-2-thiophenecarboxamide derivatives, wherein 5-chloro-N-(2-oxiranylmethyl)-2-thiophenecarboxamide (1.0 equivalent) is stirred for 2 hours to 6 hours with a primary amine or aniline derivative (1.5 equivalents to 2.5 equivalents) in the presence of a solvent at room temperature or at temperatures up to 80° C. The product can be isolated from the reaction mixture by chromatography.

PCT Publication No. WO 2012/092873 provides a process for the preparation of rivaroxaban, wherein 5-chloro-N-[(2S)-3-chloro-2-hydroxypropyl]thiophene-2-carboxamide or 5-chloro-N-[(2S)-oxiran-2-ylmethyl]thiophene-2-carboxamide is treated with substituted or unsubstituted phenyl methyl[4(3-oxo-morpholin-4-yl)phenyl]carbamate.

The prior art processes for the preparation of rivaroxaban and/or its intermediates involve long reaction times, make use of corrosive hydrobromic acid, and use expensive starting materials, catalysts, and chromatography. These processes generate corrosive hydrobromic acid as a by-product and provide products in lower yield. Accordingly, these processes are not suitable on an industrial scale.

The present inventors have developed simple, safe, efficient, economical, industrially feasible processes that provide rivaroxaban and its intermediates in good yield.

### SUMMARY OF THE INVENTION

The present invention provides processes for the preparation of rivaroxaban and its intermediates.

### DETAILED DESCRIPTION OF THE INVENTION

The present invention provides processes for the preparation of rivaroxaban and its intermediates.

A first aspect of the present invention provides a process for the preparation of 5-chloro-N-[(2S)-3-chloro-2-hydroxypropyl]thiophene-2-carboxamide of Formula II

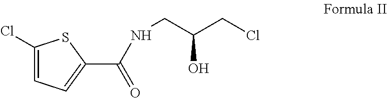 [SMILES: O=C(NC[C@H](O)CCl)c1ccc(Cl)s1]

wherein the process comprises treating a compound of Formula III

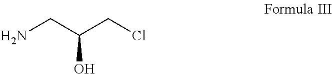

or a salt thereof with a reactive derivative of a compound of Formula IV

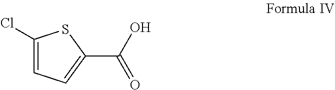

to obtain the compound of Formula II.

A second aspect of the present invention provides a process for the preparation of 5-chloro-N-[(2S)-3-chloro-2-hydroxypropyl]thiophene-2-carboxamide of Formula II,

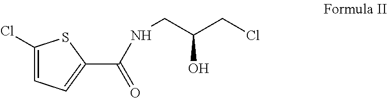 [SMILES: O=C(NC[C@H](O)CCl)c1ccc(Cl)s1]

wherein the process comprises treating a compound of Formula III

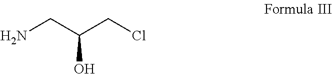

or a salt thereof with a compound of Formula IVa

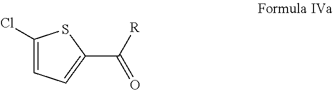

wherein R is Cl, Br, or I, to obtain the compound of Formula II.

A third aspect of the present invention provides a process for the preparation of rivaroxaban of Formula I,

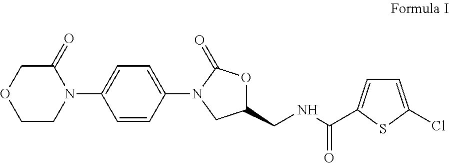

wherein the process comprises:

a) treating a compound of Formula III

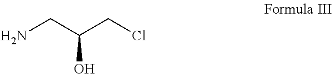

or a salt thereof with a reactive derivative of a compound of Formula IV

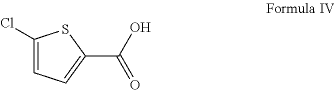

to obtain a compound of Formula II; and

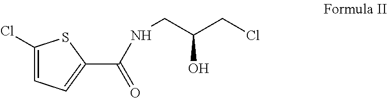 [SMILES: O=C(NC[C@H](O)CCl)c1ccc(Cl)s1]

b) converting the compound of Formula II into rivaroxaban.

A fourth aspect of the present invention provides a process for the preparation of rivaroxaban of Formula I,

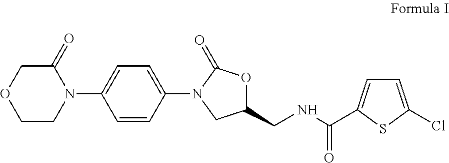

wherein the process comprises:

a) treating a compound of Formula III

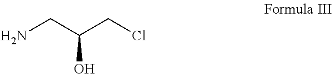

or a salt thereof with a compound of Formula IVa

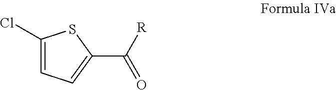

wherein R is Cl, Br, or I, to obtain a compound of Formula II; and

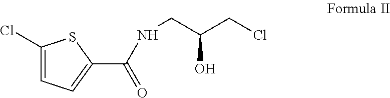 [SMILES: O=C(NC[C@H](O)CCl)c1ccc(Cl)s1]

b) converting the compound of Formula II into rivaroxaban.

A fifth aspect of the present invention provides a process for the preparation of a compound of Formula V,

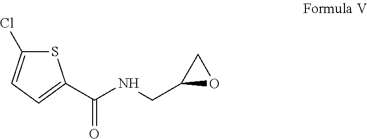 [SMILES: O=C(NC[C@H]1CO1)c1ccc(Cl)s1]

wherein the process comprises treating a compound of Formula II

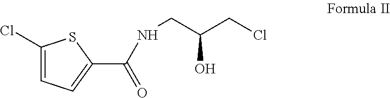 [SMILES: O=C(NC[C@H](O)CCl)c1ccc(Cl)s1]

with a base to obtain the compound of Formula V.

A sixth aspect of the present invention provides a process for the preparation of rivaroxaban of Formula I,

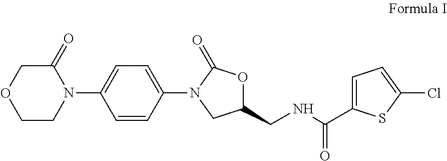

wherein the process comprises:

a) treating a compound of Formula III

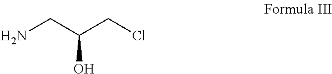

or a salt thereof with a reactive derivative of a compound of Formula IV

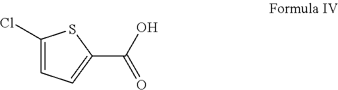

to obtain a compound of Formula II;

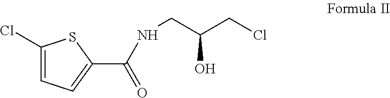 [SMILES: O=C(NC[C@H](O)CCl)c1ccc(Cl)s1]

b) treating the compound of Formula II

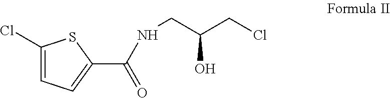 [SMILES: O=C(NC[C@H](O)CCl)c1ccc(Cl)s1]

with a base to obtain a compound of Formula V; and

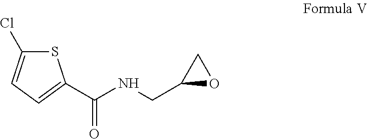 [SMILES: O=C(NC[C@H]1CO1)c1ccc(Cl)s1]

c) converting the compound of Formula V into rivaroxaban.

A seventh aspect of the present invention provides a process for the preparation of rivaroxaban of Formula I,

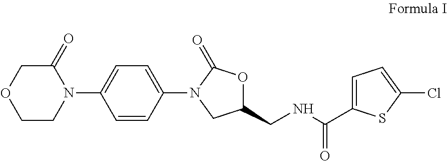

wherein the process comprises:

a) treating a compound of Formula III

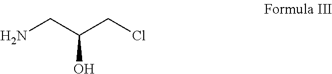

or a salt thereof with a compound of Formula IVa

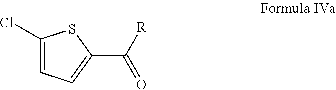

wherein R is Cl, Br, or I, to obtain a compound of Formula II;

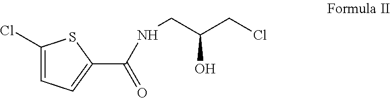 [SMILES: O=C(NC[C@H](O)CCl)c1ccc(Cl)s1]

b) treating the compound of Formula II

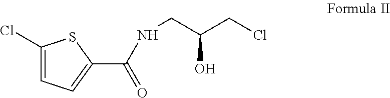 [SMILES: O=C(NC[C@H](O)CCl)c1ccc(Cl)s1]

with a base to obtain a compound of Formula V; and

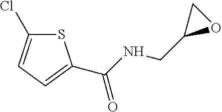 [SMILES: O=C(NC[C@H]1CO1)c1ccc(Cl)s1]

c) converting the compound of Formula V into rivaroxaban.

An eighth aspect of the present invention provides a process for the preparation of rivaroxaban of Formula I,

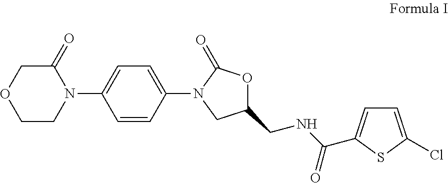

wherein the process comprises:

a) treating a compound of Formula III

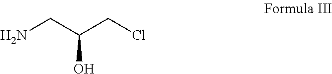

or a salt thereof with a reactive derivative of a compound of Formula IV

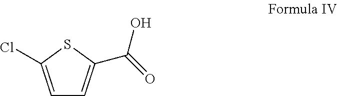

to obtain a compound of Formula II;

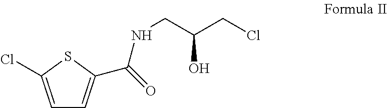 [SMILES: O=C(NC[C@H](O)CCl)c1ccc(Cl)s1]

b) treating the compound of Formula II

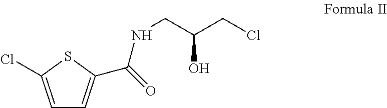 [SMILES: O=C(NC[C@H](O)CCl)c1ccc(Cl)s1]

with a base to obtain a compound of Formula V;

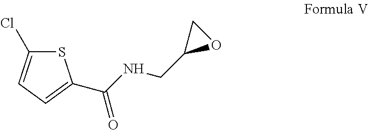 [SMILES: O=C(NC[C@H]1CO1)c1ccc(Cl)s1]

c) treating the compound of Formula V

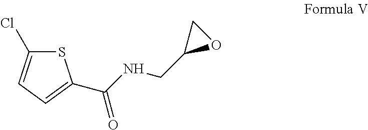 [SMILES: O=C(NC[C@H]1CO1)c1ccc(Cl)s1]

with a compound of Formula VI

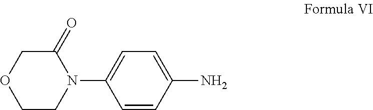 [SMILES: Nc1ccc(N2CCOCC2=O)cc1]

to obtain a compound of Formula VII; and

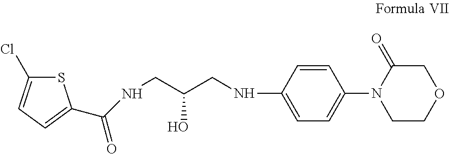 [SMILES: O=C(NC[C@@H](O)CNc1ccc(N2CCOCC2=O)cc1)c1ccc(Cl)s1]

d) converting the compound of Formula VII into rivaroxaban.

A ninth aspect of the present invention provides a process for the preparation of rivaroxaban of Formula I,

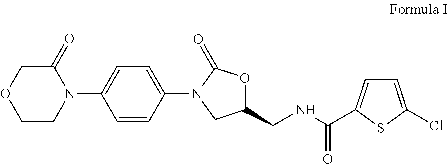

wherein the process comprises:

a) treating a compound of Formula III

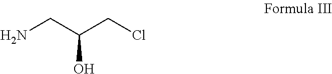

or a salt thereof with a compound of Formula IVa

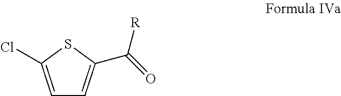

wherein R is Cl, Br, or I, to obtain a compound of Formula II;

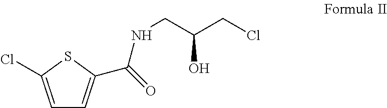 [SMILES: O=C(NC[C@H](O)CCl)c1ccc(Cl)s1]

b) treating the compound of Formula II

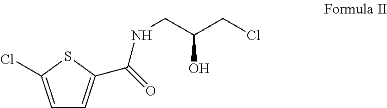 [SMILES: O=C(NC[C@H](O)CCl)c1ccc(Cl)s1]

with a base to obtain a compound of Formula V;

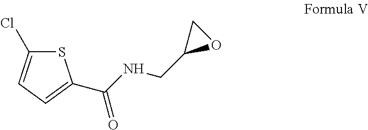 [SMILES: O=C(NC[C@H]1CO1)c1ccc(Cl)s1]

c) treating the compound of Formula V

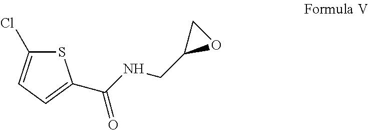 [SMILES: O=C(NC[C@H]1CO1)c1ccc(Cl)s1]

with a compound of Formula VI

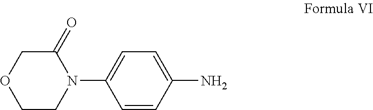 [SMILES: Nc1ccc(N2CCOCC2=O)cc1]

to obtain a compound of Formula VII; and

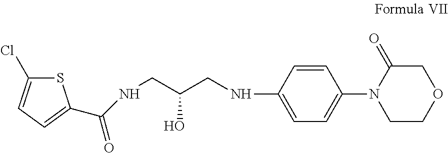 [SMILES: O=C(NC[C@@H](O)CNc1ccc(N2CCOCC2=O)cc1)c1ccc(Cl)s1]

d) converting the compound of Formula VII into rivaroxaban.

A tenth aspect of the present invention provides a compound of Formula II.

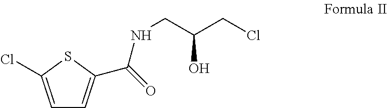 [SMILES: O=C(NC[C@H](O)CCl)c1ccc(Cl)s1]

An eleventh aspect of the present invention provides use of the compound of Formula II

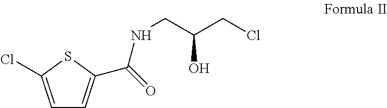 [SMILES: O=C(NC[C@H](O)CCl)c1ccc(Cl)s1]

for the preparation of rivaroxaban.

The compound of Formula III or salts thereof, the reactive derivative of the compound of Formula IV, or the compound of Formula IVa, may be prepared by any method provided in the art, for example, the methods described in U.S. Pat. No. 7,157,456, U.S. Pat. No. 6,107,519, or any analogous method. The salt of the compound of Formula III, for example the hydrochloride salt of the compound of Formula III, may also be prepared as described herein. The compound of Formula III is treated with the reactive derivative of the compound of Formula IV, for example, 5-chlorothiophene-2-carbonyl chloride, to obtain the compound of Formula II in a solvent. The reactive derivative of the compound of Formula IV may be reacted with the compound of Formula III after isolation from the reaction mixture in which it is formed, or the reaction mixture containing the reactive derivative of the compound of Formula IV can also be used for the reaction with the compound of Formula III. The reactive derivative of the compound of Formula IV is reacted with the compound of Formula III in the presence of a base. The base may be, for example, sodium bicarbonate. When a salt of the compound of Formula III, such as the hydrochloride salt is used, it may be treated with a base such as sodium bicarbonate prior to the reaction with the compound of Formula IV. The molar ratio of the base and the salt of a compound of Formula III may range from about 1:1 to about 4:1. The solvent should not interfere with the reaction, and can be selected from the group comprising tetrahydrofuran, toluene, dichloromethane, ethyl acetate, or mixtures thereof. The compound of Formula III is treated with the compound of Formula IV in the solvent at about 0° C. to about 35° C. The resulting mixture is stirred for about 1 hour to about 8 hours at about 0° C. to about 35° C. The compound of Formula II may be isolated from the mixture by methods including concentration, distillation, decantation, filtration, evaporation, centrifugation, or a combination thereof, and may further be dried.

Further, the compound of Formula II can be converted into rivaroxaban of Formula I by following the processes mentioned herein, or processes provided in prior art, for example, U.S. Pat. No. 8,106,192.

The compound of Formula II is treated with base in solvent to obtain the compound of Formula V. The solvent may be 1,4-dioxane, methanol, ethanol, or their mixtures with water. The base may be sodium carbonate, potassium carbonate, calcium carbonate, sodium hydroxide, potassium hydroxide, or mixtures thereof. The base may be used as a solid or in solution. The compound of Formula II is treated with the base at about 0° C. to about 30° C. The mixture is stirred for about 1 hour to about 8 hours at about 0° C. to about 30° C. The product may be isolated from the mixture by methods including concentration, distillation, decantation, filtration, evaporation, centrifugation, or a combination thereof.

The compound of Formula V is treated with the compound of Formula VI in a solvent to obtain the compound of Formula VII. The solvent may be ethanol, methanol, tetrahydrofuran, or their mixtures with water. The mixture containing the compound of Formula V and the compound of Formula VI is heated to reflux for about 0.5 hours to about 6 hours. The reaction mass is cooled to a temperature of about 0° C. to about 35° C. and stirred for about 0.5 hours to about 4 hours at about 0° C. to about 35° C. The compound of Formula VII may be isolated from the mixture by methods including concentration, distillation, decantation, filtration, evaporation, centrifugation, or a combination thereof, and may further be dried.

The compound of Formula VII is treated with 1,1-carbonyldiimidazole in a solvent. The solvent may be dichloromethane. The mixture is stirred for about 2 hours to about 6 hours at about 25° C. to about 30° C. The compound of Formula I may be isolated from the mixture by methods including concentration, distillation, decantation, filtration, evaporation, centrifugation, or a combination thereof, and may further be dried.

Further, the compound of Formula V can also be converted into rivaroxaban of Formula I by the processes provided in prior art, for example, PCT Publication No. WO 2011/098501 or U.S. Pat. No. 8,106,192.

The salt of a compound of Formula III in the present invention includes, for example, hydrochloride salts, hydrobromide salts, sulfate salts, nitrate salts, phosphate salts, formate salts, acetate salts, trifluoroacetate salts, methanesulfonate salts, and p-toluenesulfonate salts.

The reactive derivative of a compound of Formula IV in the present invention includes acid halides, acid azides, acid anhydrides, mixed acid anhydrides, active amides, active esters, and active thio esters. Examples of reactive derivatives include acid chloride, acid amide of a free acid, di-ethoxyphosphoric acid ester, p-nitrophenyl ester, cyanomethyl ester, pentachlorophenyl ester, N-hydroxysuccinimide ester, imidazolyl ester, N-hydroxy phthalimide ester, 1-hydroxybenzotriazole ester, 6-chloro-1-hydroxybenzotriazole ester, 1-hydroxy-1H-2-pyridone ester, 2-pyridylthiol ester, and 2-benzothiazolylthiol ester.

The term “about”, as used herein, when used along with values assigned to certain measurements and parameters means a variation of up to 10% from such values, or in case of a range of values, means up to a 10% variation from both the lower and upper limits of such ranges.

The term “ambient temperature”, as used herein, refers to a temperature in the range of 0° C. to 35° C.

While the present invention has been described in terms of its specific embodiments, certain modifications and equivalents will be apparent to those skilled in the art, and are intended to be included within the scope of the present invention.

### EXAMPLES

### Example 1

### Preparation of (2S)-1-amino-3-chloropropan-2-ol hydrochloride (Formula III)

A solution of benzaldehyde (50 g, 0.540 moles) in ethanol (100 mL) was cooled to 15° C., and aqueous ammonia (25%, 57.4 mL) was added drop wise over 15 minutes to 20 minutes. Ethanol (25 mL) was added to the mixture. The mixture was stirred at 15° C. to 20° C. for 15 minutes to 20 minutes. (S)-Epichlorohydrin (50 g, 0.540 moles) and ethanol (50 mL) were added. The reaction mixture was allowed to warm to 40° C. and stirred for 1 hour at 15° C. to 40° C. The reaction mixture was again stirred at 35° C. to 40° C. for 6 hours, cooled to 25° C. to 30° C., and further stirred for 12 hours. The solution was concentrated to dryness under vacuum at 50° C. to 55° C. Ethanol (50 mL) was added to the oil obtained, and the mixture was concentrated under vacuum at 50° C. to 55° C. Toluene (125 mL) was added to the oil obtained, and the mixture was heated to 35° C. to 40° C. Aqueous hydrochloric acid (6.8 N, 129.5 mL) was added to the solution at 35° C. to 40° C. and stirred for 2 hours. The reaction mass was cooled to 25° C. to 30° C., and the aqueous layer was separated. The organic layer was extracted with water (50 mL). The combined aqueous layers were concentrated under vacuum at 70° C. to 75° C. to get a semisolid material. The semisolid material was charged with ethanol (25 mL) and heated to 60° C. to 65° C. to get a clear solution. The solution was first cooled to 25° C. to 30° C. and then to −20° C. The slurry obtained was stirred for 1 hour at −20° C. The slurry was filtered and suck dried. The wet solid was dried at 45° C. to 50° C. under vacuum.

Yield=31.5 g (50%)

### Example 2

### Preparation of 5-chloro-N-[(2S)-3-chloro-2-hydroxypropyl]thiophene-2-carboxamide (Formula II)

Sodium bicarbonate (11.1 g, 0.132 moles) was added to a solution of (2S)-1-amino-3-chloropropan-2-ol hydrochloride (Formula III—15 g, 0.102 moles) in tetrahydrofuran (45 mL) and deionized water (90 mL) at ambient temperature. The mixture was stirred at 25° C. to 30° C. for 10 minutes to 15 minutes. The mixture was cooled to 15° C. and a solution of 5-chlorothiophene-2-carbonyl chloride (a reactive derivative of Formula IV, or Formula IVa, wherein R═Cl) (24 g, 0.132 moles) in toluene (22.5 mL) was added at 10° C. to 15° C. over 30 minutes to 35 minutes. The mixture was stirred at 10° C. to 15° C. for 2 hours, and the reaction mass was heated to 25° C. to 30° C. The organic layer was separated, and the aqueous layer was extracted with toluene (45 mL). The combined organic layers were concentrated in vacuum at 45° C. to 50° C. to get a brown colored solid. The solid was suspended in toluene (75 mL). The suspension was heated to 45° C. to 50° C. and stirred at 45° C. to 50° C. for 15 minutes. The mixture was cooled to 25° C. to 30° C., and stirred at 25° C. to 30° C. for 2 hours. The slurry obtained was filtered, washed with toluene (10 mL), and the wet solid was dried at 50° C. to 55° C. under vacuum.

Yield=19.0 g (75%)

Melting Point=107° C. to 109° C.

MS (m/z)=254

### Example 3

### Preparation of 5-chloro-N-[(2S)-oxiran-2-ylmethyl]thiophene-2-carboxamide (Formula V)

A solution of 5-chloro-N-[(2S)-3-chloro-2-hydroxypropyl]thiophene-2-carboxamide (Formula II—5.0 g, 0.0196 moles) in methanol (20 mL) was cooled to 0° C. to 5° C., and a solution of sodium hydroxide (0.787 g, 0.0196 moles) in deionized water (20 mL) was added at 0° C. to 10° C. The mixture was stirred for 5 hours at 0° C. to 5° C. The reaction mass was concentrated at 50° C. to 55° C. under vacuum. The residue obtained was suspended in dichloromethane (30 mL), and the solution was washed with deionized water (35 mL). The organic layer was separated and concentrated under vacuum at 35° C. to 40° C. to obtain the title compound.

Yield=3.5 g (80%)

### Example 4

### Preparation of 5-chloro-N-[(2R)-2-hydroxy-3-{[4-(3-oxomorpholin-4-yl)phenyl]amino}propyl]thiophene-2-carboxamide (Formula VII)

4-Aminophenyl morpholinone-3-one (Formula VI—1.76 g, 0.00914 moles) was added to a solution of 5-chloro-N-[(2S)-oxiran-2-ylmethyl]thiophene-2-carboxamide (Formula V—2 g, 0.00919 moles) in ethanol (31.5 mL) and deionized water (3.5 mL) at ambient temperature. The mixture was allowed to heat to 70° C. to 75° C. and stirred for 4 hours at 70° C. to 75° C. The reaction mixture was cooled to 15° C., and the slurry obtained was stirred for 1 hour at 15° C. to 20° C. The slurry was filtered and suck dried. The wet solid was dried under vacuum at 40° C. to 45° C.

Yield=2.9 g (77%)

### Example 5

### Preparation of 5-chloro-N-({(5S)-2-oxo-3-[4-(3-oxomorpholin-4-yl)phenyl]-1,3-oxazolidin-5-yl}methyl)thiophene-2-carboxamide (rivaroxaban of Formula I)

1,1-Carbonyldiimidazole (0.35 g, 0.00216 moles) was added to a solution of 5-chloro-N-[(2R)-2-hydroxy-3-{[4-(3-oxomorpholin-4-yl)phenyl]amino}propyl]thiophene-2-carboxamide (Formula VII—0.5 g, 0.00121 moles) in dichloromethane (7.5 mL). The mixture was stirred for 3 hours at 25° C. to 30° C. The slurry of the reaction mass was filtered, washed with dichloromethane (2.0 mL), and the wet solid was dried at 40° C. to 45° C. under vacuum.

Yield=0.45 g (85%)

We claim:

1. A process for the preparation of 5-chloro-N-[(2S)-3-chloro-2-hydroxypropyl]thiophene-2-carboxamide of Formula II, 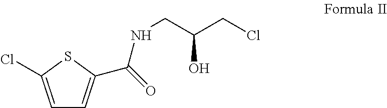 [SMILES: O=C(NC[C@H](O)CCl)c1ccc(Cl)s1]
wherein the process comprises treating a compound of Formula III 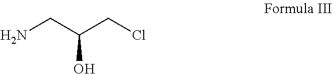
or a salt thereof with a reactive derivative of a compound of Formula IV.

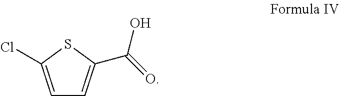

2. A process for the preparation of rivaroxaban of Formula I, 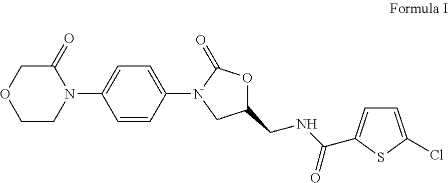
wherein the process comprises:
a) treating a compound of Formula III 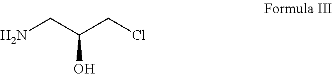
or a salt thereof with a reactive derivative of a compound of Formula IV 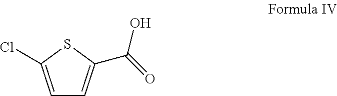
to obtain a compound of Formula II; and 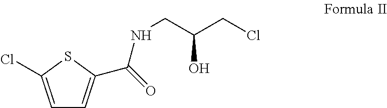 [SMILES: O=C(NC[C@H](O)CCl)c1ccc(Cl)s1]
b) converting the compound of Formula II into rivaroxaban.

3. A process for the preparation of rivaroxaban of Formula I, 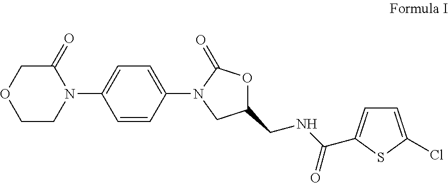
wherein the process comprises:
a) treating a compound of Formula III 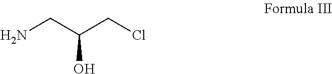
or a salt thereof with a reactive derivative of a compound of Formula IV 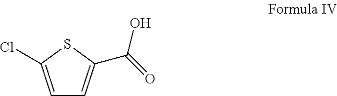
to obtain a compound of Formula II; 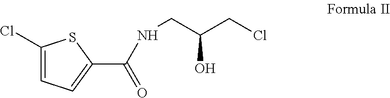 [SMILES: O=C(NC[C@H](O)CCl)c1ccc(Cl)s1]
b) treating the compound of Formula II 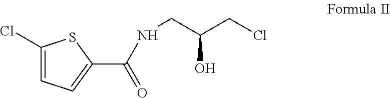 [SMILES: O=C(NC[C@H](O)CCl)c1ccc(Cl)s1]
with a base to obtain a compound of Formula V; 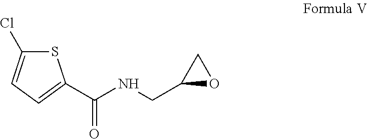 [SMILES: O=C(NC[C@H]1CO1)c1ccc(Cl)s1]
c) treating the compound of Formula V 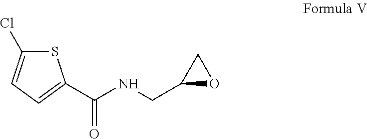 [SMILES: O=C(NC[C@H]1CO1)c1ccc(Cl)s1]
with a compound of Formula VI 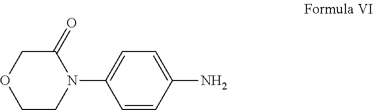 [SMILES: Nc1ccc(N2CCOCC2=O)cc1]
to obtain a compound of Formula VII; and 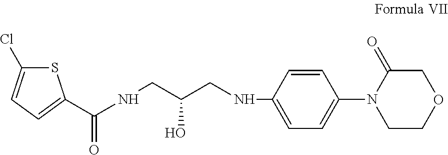 [SMILES: O=C(NC[C@@H](O)CNc1ccc(N2CCOCC2=O)cc1)c1ccc(Cl)s1]
d) converting the compound of Formula VII into rivaroxaban.

4. A process for the preparation of rivaroxaban of Formula I, 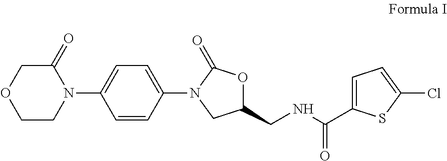
wherein the process comprises:
a) treating a compound of Formula III 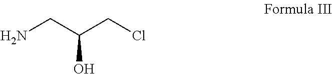
or a salt thereof with a reactive derivative of a compound of Formula IV 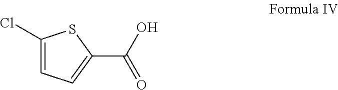
to obtain a compound of Formula II; 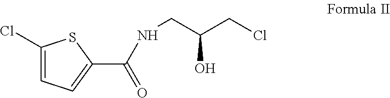 [SMILES: O=C(NC[C@H](O)CCl)c1ccc(Cl)s1]
b) treating the compound of Formula II 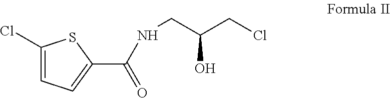 [SMILES: O=C(NC[C@H](O)CCl)c1ccc(Cl)s1]
with a base to obtain a compound of Formula V; and 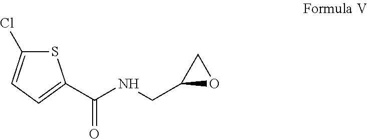 [SMILES: O=C(NC[C@H]1CO1)c1ccc(Cl)s1]
c) converting the compound of Formula V into rivaroxaban.

5. A process for the preparation of a compound of Formula V, 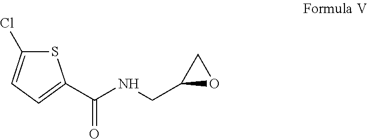 [SMILES: O=C(NC[C@H]1CO1)c1ccc(Cl)s1]
wherein the process comprises treating a compound of Formula II 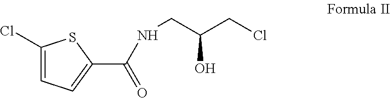 [SMILES: O=C(NC[C@H](O)CCl)c1ccc(Cl)s1]
with a base to obtain the compound of Formula V.

6. The process according to claims 3 to 5, wherein the compound of Formula II is treated with base in a solvent selected from 1,4-dioxane, methanol, ethanol, or their mixtures with water.

7. The process according to claims 3 to 6, wherein the base is selected from sodium carbonate, potassium carbonate, calcium carbonate, sodium hydroxide, potassium hydroxide, or a mixture thereof.

8. The process according to claims 1 to 4, wherein the compound of Formula III is treated with the reactive derivative of the compound of Formula IV in a solvent selected from tetrahydrofuran, toluene, dichloromethane, ethyl acetate, or a mixture thereof.

9. The process according to claim 1 to 4 or 8, wherein the compound of Formula III is treated with the reactive derivative of the compound of Formula IV in the presence of sodium bicarbonate.

10. The process according to claim 1 to 4, 8, or 9, wherein the reactive derivative of the compound of Formula IV is 5-chlorothiophene-2-carbonyl chloride, 5-chlorothiophene-2-carbonyl bromide or 5-chlorothiophene-2-carbonyl iodide.

11. The process according to claim 3, wherein the compound of Formula V is treated with the compound of Formula VI in a solvent selected from ethanol, methanol, tetrahydrofuran, or their mixture with water.

12. The process according to claim 3, wherein the compound of Formula VII is treated with 1,1-carbonyldiimidazole in dichloromethane.

13. A compound of Formula II. 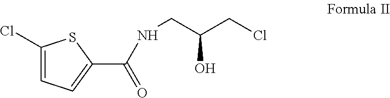 [SMILES: O=C(NC[C@H](O)CCl)c1ccc(Cl)s1]
